(19) United States
(12) Patent Application Publication
(10) Pub. No.: US 2010/0274268 A1
(43) Pub. Date: Oct. 28, 2010

(54) SQUEEZE-TO-SET MEDICAL CLAMP

(76) Inventors: ERROL O. SINGH, Upper Arlington, OH (US); Peter A. Gaydos, Hilliard, OH (US)

Correspondence Address: GALLAGHER & DAWSEY CO., L.P.A., P.O. BOX 785, COLUMBUS, OH, 43216, US

(21) Appl. No.: 12/429,315

(22) Filed: Apr. 24, 2009

(51) Int. Cl. A61B 17/122; A61B 17/00

(52) U.S. Cl. 606/157; 606/151

(57) ABSTRACT

A squeeze-to-set medical clamp having a first unset position and a second set position is disclosed. The clamp generally includes a primary arm, a secondary arm, a resilient arm connector, a top jaw, a lower jaw, and a deflector. The clamp is generally configured to receive a portion of the human or animal anatomy between the top jaw and the lower jaw when the clamp is in the second set position. In order to transition the clamp from the first unset position to the second set position a force is applied to the primary and secondary arms causing at least one of the top and lower jaws to contact and translate along the deflector until the top jaw and lower jaw are deflected. Next, the force is removed from the primary and secondary arms causing the resilient arm connector to bias the top and lower jaws toward one another.

### CROSS-REFERENCE TO RELATED APPLICATIONS

Not Applicable.

### STATEMENT REGARDING FEDERALLY SPONSORED RESEARCH OR DEVELOPMENT

Not Applicable.

### TECHNICAL FIELD

The present disclosure relates to medical devices, and more particularly, to a squeeze-to-set medical clamp.

### BACKGROUND OF THE INVENTION

Clamps are a ubiquitous tool used in the medical field for a wide range of medical procedures. Many clamps are manufactured using various materials of construction such as titanium. These clamps are typically meant to be used over a long lifespan. Such clamps are often quite expensive to manufacture and require sterilization after each use, which can further add to the cost of using the clamps. So called cross-action clamps may be manufactured with lower cost materials, but are typically shipped to the end users in a ready to use state. Shipping and storing these types of clamps in a ready to use, mechanically stressed state can compromise the clamps' effectiveness.

What is needed in the art is a clamp that is easily and cost effectively manufactured using low cost materials. Such low cost materials would allow the clamp to be used once and discarded, thus eliminating the need for expensive sterilization processes. Further, there is a need for a cross-action type clamp that is capable of being shipped to the end user in a non-stressed state, but also provides the end user with the ability to easily transition the clamp to a ready to use state. The squeeze-to-set medical clamp described herein provides a solution to these needs.

### SUMMARY OF THE INVENTION

In its most general configuration, the squeeze-to-set medical clamp advances the state of the art with a variety of new capabilities and overcomes many of the shortcomings of prior methods in new and novel ways. In its most general sense, the clamp overcomes the shortcomings and limitations of the prior art in any of a number of generally effective configurations.

Disclosed herein is a squeeze-to-set medical clamp having a first unset position and a second set position. The clamp generally includes a primary arm, a secondary arm, a resilient arm connector, a top jaw, a lower jaw, and a deflector. The clamp is generally configured to receive a portion of the human or animal anatomy, such as a penis, between the top jaw and the lower jaw when the clamp is in the second set position and ready for use. However, the clamp must first be transitioned for the first unset position to the second set position.

In order to transition the clamp from the first unset position to the second set position, the following steps are taken. First, a compressive force is applied to the primary arm and the secondary arm such that at least one of the top jaw and lower jaw contact and translate along the deflector until the top jaw and lower jaw are deflected and a top jaw contact surface and a lower jaw contact surface are in adjacent facing relationship to each other. Next, the compressive force is removed from the primary arm and the secondary arm such that the resilient arm connector biases the top jaw and the lower jaw toward one another.

In a particular embodiment, the clamp further includes a pinch deterrent portion that prevents a portion of the anatomy desired to be clamped from passing beyond the intended clamping position. In doing so, the pinch deterrent portion, in cooperation with the top jaw and the lower jaw, create a partially bounded circumference.

In another embodiment, the clamp further includes a top jaw stop ledge having a ledge width. The top jaw stop ledge effectively serves as a safety feature by engaging a portion of the secondary arm to prevent the clamp from being unintentionally moved from the second set position to the first unset position.

Numerous alterations, modifications, and variations of the preferred embodiments disclosed herein will be apparent to those skilled in the art and they are all anticipated and contemplated to be within the spirit and scope of the clamp.

### BRIEF DESCRIPTION OF THE DRAWINGS

Without limiting the scope of the present squeeze-to-set medical clamp as claimed below and referring now to the drawings and figures:

FIG. 1 shows an elevation view of an embodiment of a squeeze-to-set medical clamp in a first unset position, not to scale;

FIG. 2 shows an elevation view of an embodiment of a squeeze-to-set medical clamp in a second set position, not to scale;

FIG. 3 shows an elevation view of an embodiment of a squeeze-to-set medical clamp in a first unset position, not to scale;

FIG. 4 shows an elevation view of an embodiment of a squeeze-to-set medical clamp in a second set position, not to scale;

FIG. 5 shows an elevation view of an embodiment of a squeeze-to-set medical clamp in a receiving position, not to scale;

FIG. 6 shows an elevation view of an embodiment of a squeeze-to-set medical clamp in a first unset position, not to scale;

FIG. 7 shows a perspective view of an embodiment of a squeeze-to-set medical clamp in a first unset position, not to scale;

FIG. 8 shows a perspective view of an embodiment of a squeeze-to-set medical clamp in a first unset position, not to scale;

FIG. 9 shows an elevation view of an embodiment of a squeeze-to-set medical clamp in a first unset position, not to scale;

FIG. 10 shows an elevation view of an embodiment of a squeeze-to-set medical clamp in a first unset position, not to scale;

FIG. 11 shows a bottom plan view of an embodiment of a squeeze-to-set medical clamp in a first unset position, not to scale;

FIG. 12 shows a top plan view of an embodiment of a squeeze-to-set medical clamp in a first unset position, not to scale;

FIG. 13 shows an elevation view of an embodiment of a squeeze-to-set clamp with a penis positioned between a top jaw and a lower jaw, not to scale; and

FIG. 14 shows an elevation view of an embodiment of a squeeze-to-set clamp exerting a clamping force on a penis, not to scale.

These drawings are provided to assist in the understanding of the exemplary embodiments of the squeeze-to-set medical clamp as described in more detail below and should not be construed as unduly limiting the squeeze-to-set medical clamp. In particular, the relative spacing, positioning, sizing and dimensions of the various elements illustrated in the drawings are not drawn to scale and may have been exaggerated, reduced or otherwise modified for the purpose of improved clarity. Those of ordinary skill in the art will also appreciate that a range of alternative configurations have been omitted simply to improve the clarity and reduce the number of drawings.

### DETAILED DESCRIPTION OF THE INVENTION

The claimed squeeze-to-set medical clamp (100) enables a significant advance in the state of the art. The preferred embodiments of the clamp (100) accomplish this by new and novel arrangements of elements and methods that are configured in unique and novel ways and which demonstrate previously unavailable but preferred and desirable capabilities. The description set forth below in connection with the drawings is intended merely as a description of the presently preferred embodiments of the clamp (100), and is not intended to represent the only form in which the clamp (100) may be constructed or utilized. The description sets forth the designs, functions, means, and methods of implementing the clamp (100) in connection with the illustrated embodiments. It is to be understood, however, that the same or equivalent functions and features may be accomplished by different embodiments that are also intended to be encompassed within the spirit and scope of the claimed clamp (100).

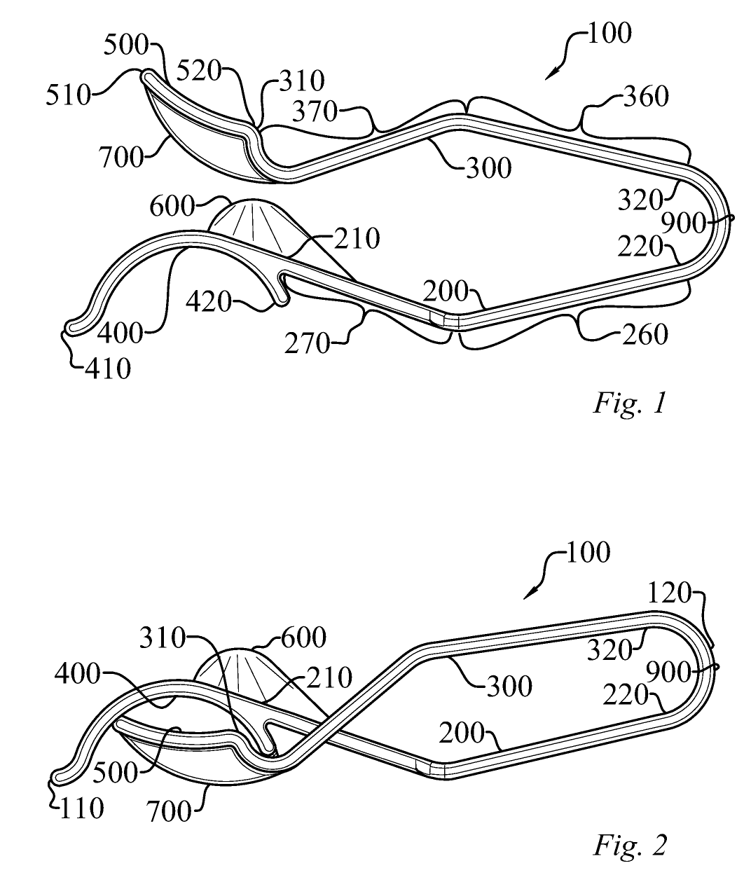

With reference generally to FIGS. 1-12, a squeeze-to-set medical clamp (100) is shown. The clamp (100) is designed and configured to cradle and clamp a portion of the human or animal anatomy. The embodiments throughout reference a urological application for a penis (P); however, one skilled in the art will appreciate that the clamp (100) may be used on other parts of the anatomy. By way of example, and not limitation, the clamp (100) may be advantageously used in urological applications whereby the clamp (100) exerts pressure against the urethra (U) to effectively prevent the passage of fluid.

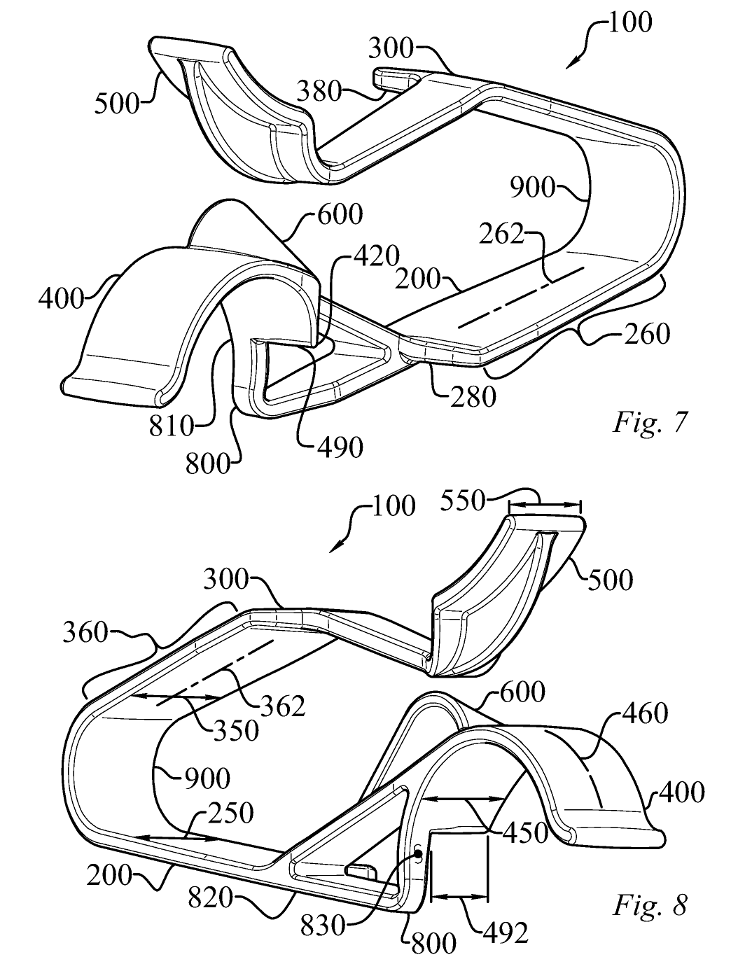

Referring now to FIGS. 1 and 2, the clamp (100) has a clamp distal end (110) and a clamp proximal end (120). In FIG. 1, the clamp (100) is in a first unset position, and in FIG. 2, the clamp (100) is in a second set position. The clamp (100) generally includes a primary arm (200), a secondary arm (300), a top jaw (400), a lower jaw (500), a deflector (600), and a resilient arm connector (900). The primary arm (200) includes a primary arm distal end (210), a primary arm proximal end (220), a primary arm width (250), seen only in FIG. 8, a primary arm gripping portion (260), a primary jaw connector portion (270), and a primary arm opening stop guide (280), which is best seen in FIG. 7. The secondary arm (300) includes a secondary arm distal end (310), a secondary arm proximal end (320), a secondary arm width (350), seen only in FIG. 8, a secondary arm gripping portion (360), a secondary jaw connector portion (370), and a secondary arm opening stop guide (380), which is also best seen in FIG. 7.

The resilient arm connector (900) joins the primary arm (200) at the primary arm proximal end (220) to the secondary arm (300) at the secondary arm proximal end (320). The resilient arm connector (900) provides a spring force to enable the clamp (100) to perform a clamping action. Although the clamp (100) is shown as a single molded piece throughout FIGS. 1-12, it should be noted that the clamp (100) may be comprised of separate and distinct components. For example, the resilient arm connector (900) may be formed of a material that is different from other clamp (100) components.

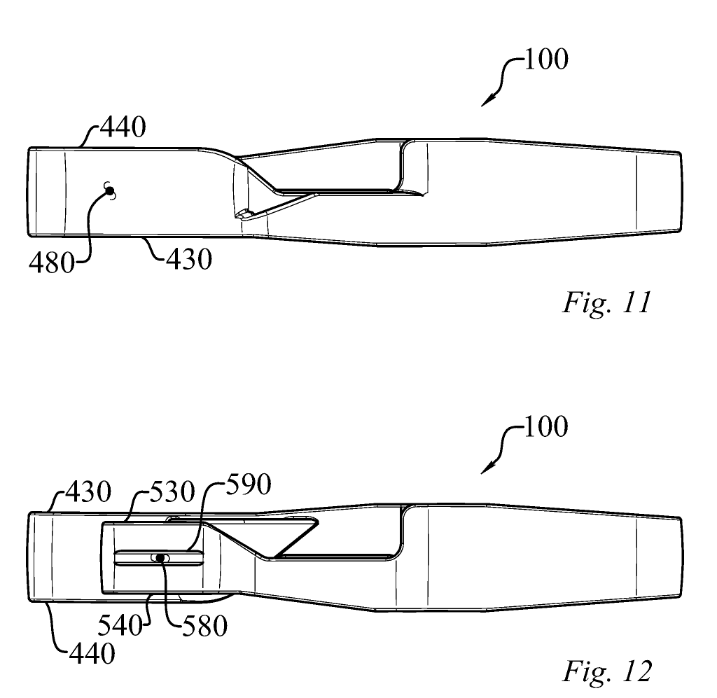

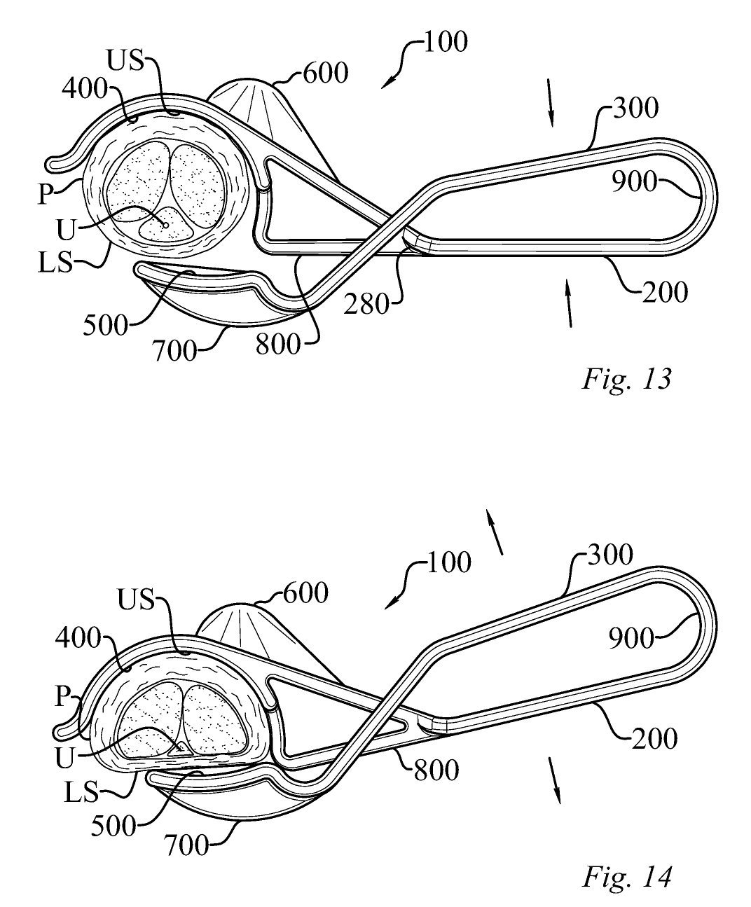

With general reference to FIGS. 1, 2, 8, 9, 11, and 12, the top jaw (400) is joined to the primary arm (200) by the primary jaw connector portion (270) and includes a top jaw distal end (410), a top jaw proximal end (420), a top jaw dextral side (430), a top jaw sinistral side (440), a top jaw width (450), a top jaw centerline (460), a top jaw radius of curvature (470), which is best seen in FIG. 6, and a top jaw contact surface (480), which is best seen in FIG. 11. The lower jaw (500) is joined to the secondary arm (300) by the secondary jaw connector portion (370) and includes a lower jaw distal end (510), a lower jaw proximal end (520), a lower jaw dextral side (530), a lower jaw sinistral side (540), a lower jaw width (550), a lower jaw centerline (560), a lower jaw radius of curvature (570), which is best seen in FIG. 6, and a lower jaw contact surface (580), which is best seen in FIG. 12. As seen in FIGS. 13 and 14, the top jaw (400) and lower jaw (500) are sized and dimensioned to cradle a portion of the human or animal anatomy, such as the upper surface (US) and lower surface (LS) of a penis (P), respectively.

Referring now to FIGS. 1, 2, 9 and 10, the clamp (100) includes a deflector (600) positioned on at least one of the top jaw (400) and lower jaw (500). The deflector (600) has a deflector height (610), a deflector width (620), and a deflector angle (630). As discussed in more detail below, the deflector (600) aids in transitioning the clamp (100) from the first unset position to the second set position. Further, the clamp (100) may include more than one deflector (600) and in this embodiment the deflectors (600) may be placed on opposite jaws (400, 500) to cooperate with each other.

As seen in FIG. 1, the clamp (100) is in the first unset position. In the first unset position the clamp (100) is not ready for use. Thus, the clamp (100) must be transitioned from the first unset position to the second set position to put the clamp (100) in a ready to use state. The transition from the first unset position to the second set position may be accomplished by a two-step process. First, a user applies a compressive force to the primary arm (200) and the secondary arm (300) by positioning their fingers on the primary arm gripping portion (260) and the secondary arm gripping portion (360) and squeezing the primary arm (200) towards the secondary arm (300). The compressive force will cause at least one of the top jaw (400) and lower jaw (500) to contact and translate along the deflector (600) until the top jaw (400) and lower jaw (500) are deflected and the top jaw contact surface (480) and the lower jaw contact surface (580) are in adjacent facing relationship to each other. Thus, the deflector (600) acts as a bearing surface that aids in deflecting the top jaw (400) and the lower jaw (500) until they bypass one another and the top jaw contact surface (480) and the lower jaw contact surface (580) are in adjacent facing relationship.

Next, the user removes the compressive force applied to the primary arm (200) and the secondary arm (300). The removal of the compressive force causes the resilient arm connector (900) to bias the top jaw (400) and the lower jaw (500) toward one another, thus putting the clamp (100) in the second set position. When transitioning the clamp (100) from the first unset position to the second set position, the clamp (100) provides feedback in the form of a noticeable “click” sound after the jaws (400, 500) have bypassed each other and return to their natural horizontal positions. When the “click” is heard, the user will know that the clamp (100) is in the second set position and ready for use. Thus, users who are visually impaired will be capable of knowing that the clamp (100) is in the second set position and ready for use.

In one embodiment, the amount of compressive force applied to the primary arm (200) and the secondary arm (300) required to transition the clamp (100) from the first unset position to the second set position is in a range of about 3.00 lbf to about 6.00 lbf. Such a range allows the clamp (100) to be easily transitioned from the first unset position to the second set position by virtually any user, even those with arthritis of the finger joints.

After the clamp (100) is transitioned to the second set position, the clamp (100) is now ready for use. A user may separate the top jaw (400) from the lower jaw (500) by applying a compressive force to the primary arm (200) and the secondary arm (300) by positioning their fingers on the primary arm gripping portion (260) and the secondary arm gripping portion (360) and squeezing the primary arm (200) towards the secondary arm (300). When the top jaw (400) is separated from the lower jaw (500) the clamp (100) is in a receiving position capable of receiving a penis (P), as seen in FIG. 13, or another part of the human or animal anatomy. After the penis (P) is in the desired position between the top jaw (400) and the lower jaw (500), the user removes the compressive force causing the resilient arm connector (900) to bias the top jaw (400) and lower jaw (500) towards one another to provide a clamping force on the penis (P), as seen in FIG. 14.

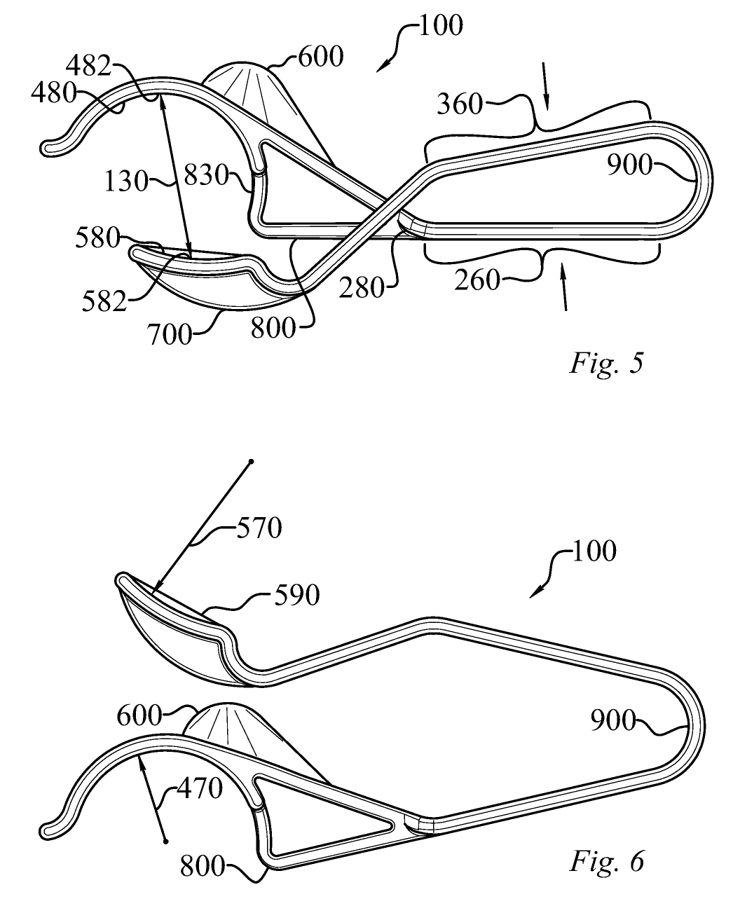

Referring now to FIG. 5, the clamp (100) includes a clamp opening (130). The clamp opening (130) is defined by the distance between a top jaw contact surface center (482) and a lower jaw contact surface center (582). One with skill in the art will recognize that the clamp opening (130) is variable and dependent on the amount of compressive force applied to the primary arm (200) and the secondary arm (300). Similarly, the amount of clamping force exerted by the clamp (100) is variable and dependent on the clamp opening (130). For example, when the clamp (100) is in the second set position, and thus the minimum clamp opening, the clamping force exerted is a minimum clamping force. Similarly, when the clamp opening (130) is a maximum clamp opening, such as seen in FIG. 5, the clamping force exerted is a maximum clamping force. The maximum clamp opening is restricted by the primary arm opening stop guide (280) and the secondary arm opening stop guide (380). In use, when a compressive force is applied to the primary arm (200) and the secondary arm (300), the primary jaw connector portion (270) will contact the secondary arm opening stop guide (380) and the secondary jaw connector portion (370) will contact the primary arm opening stop guide (280), thus preventing the top jaw (400) and the lower jaw (500) from separating any further beyond the maximum clamp opening position.

In one embodiment, the minimum clamping force is at least ten percent of the maximum clamping force. This percentage ensures that the minimum clamping force will be capable of obstructing the urethra (U) of a penis (P) that fits between the top jaw (400) and the lower jaw (500) when the clamp (100) is in a position at or near the minimum clamp opening, or second set position.

In another embodiment, the clamp (100) in the second set position exerts a clamping force in a range of about 0.15 lbf to about 0.30 lbf. This range provides adequate clamping force to obstruct the urethra (U) of a penis (P) that narrowly fits between the top jaw (400) and the lower jaw (500) when the clamp (100) is at or near the second set position.

In one particular embodiment, the clamp opening (130) has a range of about 0.20 inch to about 1.80 inches. Thus, the clamp (100) effectively accommodates a wide range of penis sizes. Additionally, within the clamp opening (130) range of about 0.20 inch to about 1.80 inches, the clamp (100) exerts a clamping force in a range of about 0.15 lbf to about 1.40 lbf. Such a range provides a clamping force capable of obstructing the urethra (U) of various sized penises. However, the range also provides a clamping force that is effective for obstructing the urethra (U) without providing excessive force, which results in a range of more comfortable clamping forces. In still another embodiment, when the clamp opening (130) is about 1.00 inch, the clamp (100) exerts a clamping force of about 1.00 lbf. The above values and ranges of clamping force are attributable to the engineered design of the resilient arm connector (900). Changes in the material of construction or the specific geometry of the resilient arm connector will affect the values and ranges of the clamping force.

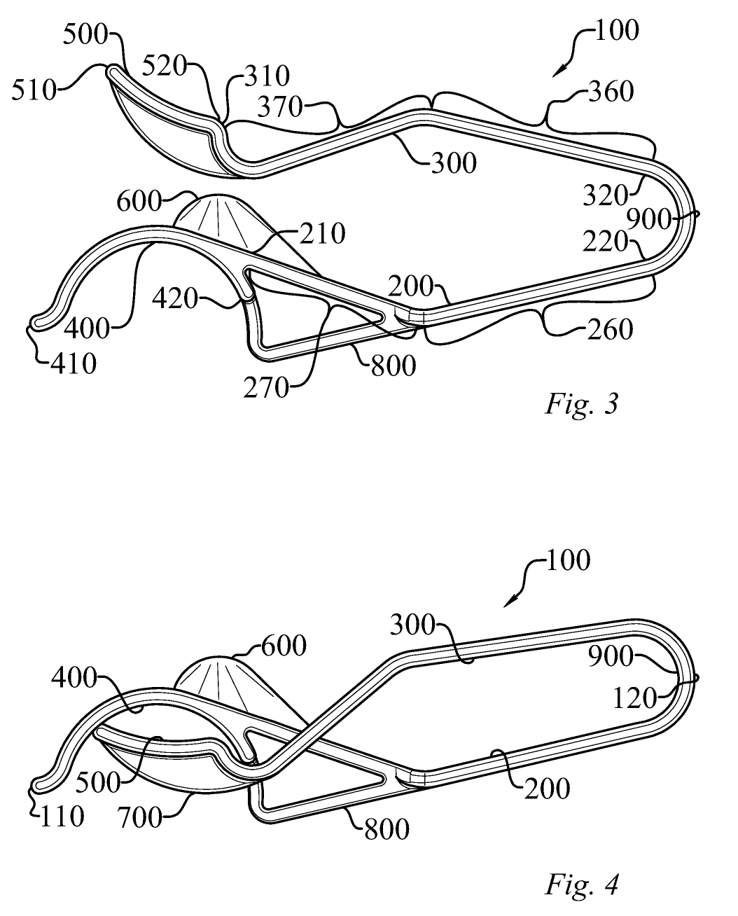

In yet another embodiment, the clamp (100) includes a pinch deterrent portion (800), as seen in FIGS. 3-8. The pinch deterrent portion (800) has a pinch deterrent portion distal end (810), a pinch deterrent portion proximal end (820), and a pinch deterrent portion contact surface (830). The pinch deterrent portion distal end (810) is joined to the top jaw proximal end (420). Further, the pinch deterrent portion proximal end (820) may be joined to the primary arm (200) to further increase its strength and rigidity. Although, the pinch deterrent portion (800) is shown as being joined to the top jaw (400) and the primary arm (200), the pinch deterrent portion (800) could be joined to the lower jaw (500) and the secondary arm (300) provided the lower jaw (500) and secondary arm (300) are designed to accommodate the necessary motion. As its name suggests, the pinch deterrent portion (800) is effective for preventing a penis (P) from getting unintentionally pinched between the primary arm (200) and the secondary arm (300). As illustrated in FIGS. 5, 13 and 14, in one embodiment, the top jaw contact surface (480), the lower jaw contact surface (580), and the pinch deterrent portion contact surface (830) form a partially bounded circumference totaling at least 225 degrees when the clamp (100) is at a maximum clamp opening. The partially bounded circumference of at least 225 degrees created by the surfaces (480, 580, 830) when the clamp (100) is at a maximum clamp opening ensures that a penis (P) will not pass through the intended clamping position and get pinched between the primary arm (200) and the secondary arm (300). It should also be noted that the pinch deterrent portion (800) may be an extension of the top jaw (400), meaning that the pinch deterrent portion (800) does not have to be joined to the primary arm (200). Similarly, the pinch deterrent portion (800) may be an extension of the lower jaw (500) without being joined to the secondary arm (300).

Referring now to FIGS. 6, 10, and 12, in still another embodiment, the clamp (100) includes a urethral rib (590). The urethral rib (590) is positioned on the lower jaw (500) and provides a raised structure for contacting the lower surface (LS) of the penis (P) to more effectively obstruct the urethra (U). Although the urethral rib (590) is shown as being flat in the drawings, the urethral rib (590) may have a concave shape or a convex shape.

With reference now to FIGS. 7 and 8, another embodiment of the clamp (100) is shown. In this embodiment, the top jaw (400) includes a top jaw stop ledge (490) having a ledge width (492). The top jaw stop ledge (490) effectively serves as a safety feature. In operation, the top jaw stop ledge (490) engages a portion of the secondary arm (300) when the clamp (100) is in the second set position to prevent unintentional movement of the clamp (100) from the second set position to the first unset position. With the top jaw stop ledge (490) engaging the secondary arm (300), a user must intentionally cause the secondary arm (300) to overcome the ledge width (492) to move the clamp (100) from the second set position to the first unset position.

As depicted throughout the drawings, the clamp (100) has a relatively constant top jaw width (450) and a relatively constant lower jaw width (550). However, the top jaw and lower jaw widths (450, 550) may vary from the jaws' proximal ends (420, 520) to the jaws' distal ends (410, 510). Such variation in jaw widths (450, 550) creates a maximum and minimum top jaw width (450) and a maximum and minimum lower jaw width (550). Providing a clamp (100) with a minimum top jaw width and lower width (450, 550) at the jaws' distal ends (410, 510) allows the clamp (100) to more easily receive a penis (P) between the top jaw (400) and the lower jaw (500).

Referring again to FIGS. 7 and 8, in one embodiment, the clamp (100) includes a ledge width (492) that is at least two-thirds of a maximum top jaw width (450). With this particular ratio of ledge width (492) to maximum top jaw width (450), it will be difficult for the clamp (100) to be unintentionally moved from the second set position to the first unset position, thus further increasing the safety of the clamp (100).

In one particular embodiment, the clamp (100) has a lower jaw width (550) that is at least thirty percent of the top jaw width (450). This percentage also includes the maximum and minimum jaw widths (450, 550). Thus, the minimum lower jaw width (550) is at least thirty percent of the minimum top jaw width (450), and the maximum lower jaw width (550) is at least thirty percent of the maximum top jaw width (450). It is preferable for the top jaw width (450) to be larger than the lower jaw width (550) because the top jaw (400) cradles the penis (P) more so than the lower jaw (500). Such a relationship between jaw widths (450, 550) allows distribution of the clamping force over a larger area by the top jaw (400) to increase comfort. Similarly, the relationship allows the clamping force to be concentrated by the lower jaw (500) to better aid in obstructing a urethra (U) when the clamp (100) is used for urological applications.

Referring now to FIG. 6, the top jaw (400) and lower jaw (500) are complementally configured for comfortably receiving a penis (P). In one embodiment, the top jaw (400) has a top jaw radius of curvature (470) in a range of about 0.50 inch to about 0.75 inch. As seen in FIG. 6, the lower jaw (500) has a lower jaw radius of curvature (570) that is larger than the top jaw radius of curvature (470). In fact, in one particular embodiment, the lower jaw radius of curvature (570) is at least forty percent larger than the top jaw radius of curvature (470). This particular embodiment ensures that the top jaw (400) contacts and cradles more of the penis (P) than the lower jaw (500). Thus, the relationship between the top jaw radius of curvature (470) and the lower jaw radius of curvature (570) allow the clamp (100) to be utilized over a wide range of penis (P) sizes.

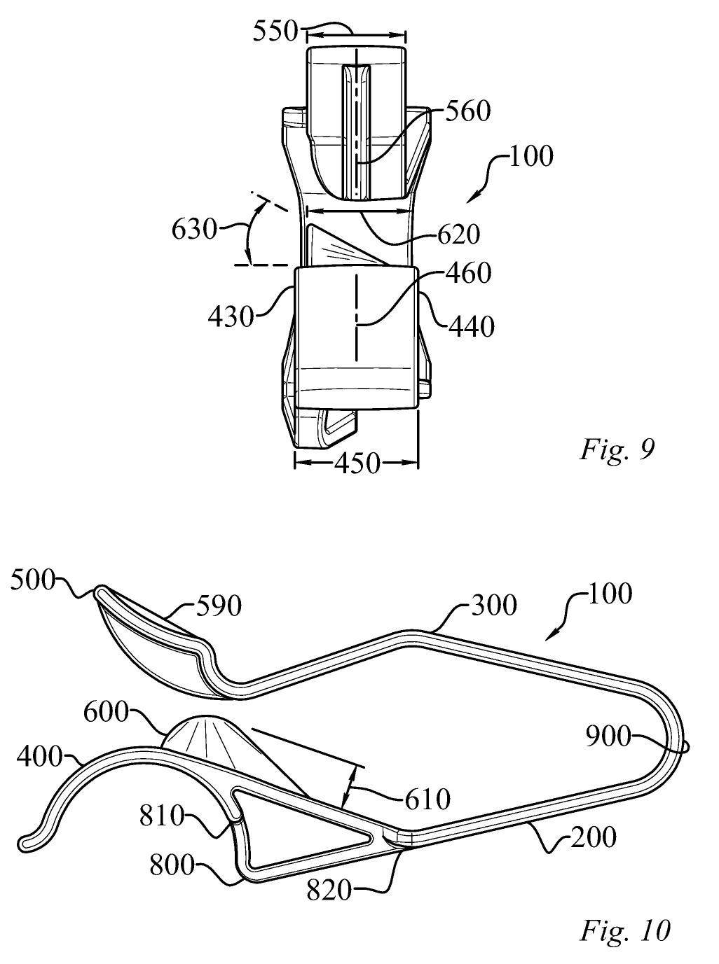

With reference now to FIGS. 9 and 10, the deflector (600) will now be described in greater detail. As mentioned above, the deflector (600) acts as a bearing surface that aids in the transitioning of the clamp (100) from the first unset position to the second set position. Throughout the drawings the deflector (600) is depicted as having a somewhat semi-conical shape. However, the deflector (600) may be formed with virtually any geometric shape so long as the deflector (600) is capable of functioning as a bearing surface to aid the transition of the clamp (100) from the first unset position to the second set position.

In one embodiment, the deflector (600) has a deflector angle (630) of at least 30 degrees. It has been found that a deflector angle (630) of at least 30 degrees enables the clamp (100) to be easily transitioned from the first unset position to the second set position with a manageable amount of compressive force applied to the primary and secondary arms (200, 300). Increasing the deflector angle (630) will allow the transition to occur with a smaller amount of compressive force, while decreasing the deflector angle (630) will require a greater amount of compressive force to make the transition. Further, a user familiar with the motion of the jaws (400, 500) during the transition between the first unset position to the second set position will often provide a small side load, or twisting motion, to assist with the translation of the jaws (400, 500) along the deflector (600). Additionally, the deflector (600) may be provided with a low friction coating or a low friction surface texture to promote reduced friction to more easily transition the clamp (100) from the first unset position to the second set position.

As seen throughout the drawings, the deflector (600) is positioned on the top jaw (400). However, the deflector (600) may be positioned on either the top jaw (400) or the lower jaw (500) so long as the deflector (600) can properly function to aid the transition of the clamp (100) from the first unset position to the second set position. In one particular embodiment, the deflector (600) is positioned on the jaw (400, 500) having the largest jaw width (450, 550).

In one embodiment, when the deflector (600) is positioned on the top jaw (400), the deflector width (620) extends from the top jaw sinistral side (440) to at least the lower jaw centerline (560). In this relationship, the deflector (600) will always extend at least half-way across the lower jaw (500) to ensure that a portion of the lower jaw (500) contacts the deflector (600) to easily transition the clamp (100) from the first unset position to the second set position. The same, but opposite, relationship also applies for an embodiment having the deflector (600) positioned on the lower jaw (500).

Along those same lines, in another embodiment, when the deflector (600) is positioned on the top jaw (400), the deflector width (620) is greater than or equal to a maximum lower jaw width (550). Thus, in this particular embodiment, the relationship between the deflector width (620) and the lower jaw width (550) is such that a portion of the lower jaw (500) will always be in contact with the deflector (600) when the clamp (100) is transitioned from the first unset position to the second set position.

As discussed above, the clamp (100) may include a top jaw stop ledge (490) having a ledge width (492). In one particular embodiment, the ledge width (492) is greater than or equal to the deflector width (620). This particular relationship ensures that the deflector (600) and the top jaw stop ledge (490) may properly perform their intended functions regardless of the size of the lower jaw (500). Furthermore, the relationship provides the clamp (100) with some flexibility at the pinch deterrent portion (800), as seen in FIGS. 7 and 8, to make the transition from the first unset position to the second set position easier.

The clamp (100) may be provided with various surface textures on various clamp (100) components. For example, the primary arm (200) and secondary arm (300), especially the primary and secondary arm gripping portions (260, 360), may include a high friction coating or high friction surface texture to promote slip resistance. Moreover, the top jaw contact surface (480) and the lower jaw contact surface (580), and in certain embodiments, the pinch deterrent portion contact surface (830), may be provided with a coating that promotes cushioning and slip resistance between the surfaces (480, 580, 830) and a penis (P).

It may thus be appreciated that the squeeze-to-set medical clamp (100) offers substantial advantages. For example, the clamp (100) may be easily and cost effectively manufactured by well known molding processes. Preferably the clamp (100) is formed of a thermoplastic polymer, such as, by way of example only and not limitation, polycarbonate, polyacrylates, fluoroplastics, acrylonitrile butadiene styrene, polyolefins, and others. It should be noted that the clamp (100) material of construction should have a sufficient modulus of elasticity and toughness to withstand the compressive force required to transition the clamp (100) from the first unset position to the second set position. Furthermore, the relatively low cost associated with manufacturing the clamp (100) allows the clamp (100) to be disposable. Thus, the clamp (100) eliminates the costs associated with sterilization after use. Additionally, the clamp (100) may be shipped to end users in the first unset position. Because the clamp (100) is formed of a thermoplastic material, shipping the clamps (100) in the second set position may create problems. For example, depending on the temperature and duration of storage, varying amounts of creep relaxation may occur in the clamps (100). This introduction of creep relaxation could result in the clamps (100) exerting highly variable clamping forces, which could reduce the effectiveness of the clamp's (100) operation. Finally, the clamp (100) structure allows the end user to squeeze the clamp (100) once to put the clamp (100) in a ready to use state.

Numerous alterations, modifications, and variations of the preferred embodiments disclosed herein will be apparent to those skilled in the art and they are all anticipated and contemplated to be within the spirit and scope of the clamp (100). For example, although specific embodiments have been described in detail, those with skill in the art will understand that the preceding embodiments and variations can be modified to incorporate various types of substitute and or additional or alternative materials, relative arrangement of elements, and dimensional configurations. Accordingly, even though only few variations of the clamp (100) are described herein, it is to be understood that the practice of such additional modifications and variations and the equivalents thereof, are within the spirit and scope of the clamp (100) as defined in the following claims. Further, dextral and sinistral sides were randomly choosen and may be reversed and remain within the scope of the clamp (100). Similarly, while the figures illustrate the jaws (400, 500) translating in one direction, the deflector (600) may be reversed and translation may occur in the opposite direction. The corresponding structures, materials, acts, and equivalents of all means or step plus function elements in the claims below are intended to include any structure, material, or acts for performing the functions in combination with other claimed elements as specifically claimed.

We claim:

1. A squeeze-to-set medical clamp (100) having a first unset position and a second set position, comprising:
a) a primary arm (200) having a primary arm distal end (210), a primary arm proximal end (220), a primary arm width (250), a primary arm gripping portion (260), a primary jaw connector portion (270), and a primary arm opening stop guide (280);
b) a secondary arm (300) having a secondary arm distal end (310), a secondary arm proximal end (320), a secondary arm width (350), a secondary arm gripping portion (360), a secondary jaw connector portion (370), and a secondary arm opening stop guide (380);
c) a resilient arm connector (900) joining the primary arm (200) at the primary arm proximal end (220) to the secondary arm (300) at the secondary arm proximal end (320);
d) a top jaw (400) joined to the primary arm (200) by the primary jaw connector portion (270), wherein the top jaw (400) includes a top jaw distal end (410), a top jaw proximal end (420), a top jaw dextral side (430), a top jaw sinistral side (440), a top jaw width (450), a top jaw centerline (460), a top jaw radius of curvature (470), and a top jaw contact surface (480) having a top jaw contact surface center (482);
e) a lower jaw (500) joined to the secondary arm (300) by the secondary jaw connector portion (370), wherein the lower jaw (500) includes a lower jaw distal end (510), a lower jaw proximal end (520), a lower jaw dextral side (530), a lower jaw sinistral side (540), a lower jaw width (550), a lower jaw centerline (560), a lower jaw radius of curvature (570), and a lower jaw contact surface (580) having a lower jaw contact surface center (582);
f) a clamp opening (130) defined by the distance between the top jaw contact surface center (482) and the lower jaw contact surface center (582); and
g) a deflector (600) positioned on at least one of the top jaw (400) and lower jaw (500), wherein the deflector (600) has a deflector height (610), a deflector width (620), and a deflector angle (630); and
wherein the squeeze-to-set medical clamp (100) is transitioned from the first unset position to the second set position by the steps of:
(i) applying a compressive force to the primary arm (200) and the secondary arm (300) such that at least one of the top jaw (400) and lower jaw (500) contact and translate along the deflector (600) until the top jaw (400) and lower jaw (500) are deflected and the top jaw contact surface (480) and the lower jaw contact surface (580) are in adjacent facing relationship to each other; and
(ii) removing the compressive force applied to the primary arm (200) and the secondary arm (300) such that the resilient arm connector (900) biases the top jaw (400) and the lower jaw (500) toward one another.

2. The squeeze-to-set medical clamp (100) of claim 1, further including a pinch deterrent portion (800) having a pinch deterrent portion distal end (810), a pinch prevention proximal end (820), and a pinch deterrent portion contact surface (830), wherein the pinch deterrent portion distal end (810) is joined to the top jaw proximal end (420), and wherein the top jaw contact surface (480), the lower jaw contact surface (580), and the pinch deterrent portion contact surface (830) form a partially bounded circumference totaling at least 225 degrees when the squeeze-to-set medical clamp (100) is at a maximum clamp opening (130).

3. The squeeze-to-set medical clamp (100) of claim 1, wherein the deflector angle (630) is at least 30 degrees.

4. The squeeze-to-set medical clamp (100) of claim 1, wherein the top jaw (400) further includes a top jaw stop ledge (490) having a ledge width (492), wherein the top jaw stop ledge (490) engages a portion of the secondary arm (300) to prevent unintentional movement of the squeeze-to-set medical clamp (100) from the second set position to the first unset position.

5. The squeeze-to-set medical clamp (100) of claim 4, wherein the ledge width (492) is at least two-thirds of a maximum top jaw width (450).

6. The squeeze-to-set medical clamp (100) of claim 1, wherein the deflector (600) is positioned on the top jaw (400) and the deflector width (620) extends from the top jaw sinistral side (440) to at least the lower jaw centerline (560).

7. The squeeze-to-set medical clamp (100) of claim 6, wherein the deflector width (620) is greater than or equal to a maximum lower jaw width (550).

8. The squeeze-to-set medical clamp (100) of claim 1, wherein the compressive force applied to the primary arm (200) and the secondary arm (300) to transition the squeeze-to-set medical clamp (100) from the first unset position to the second set position is in a range of about 3.00 lbf to about 6.00 lbf.

9. The squeeze-to-set medical clamp (100) of claim 1, wherein the squeeze-to-set medical clamp (100) in the second set position exerts a clamping force in a range of about 0.15 lbf to about 0.30 lbf.

10. The squeeze-to-set medical clamp (100) of claim 1, wherein the top jaw radius of curvature (470) is in a range of about 0.50 inch to about 0.75 inch.

11. The squeeze-to-set medical clamp (100) of claim 1, wherein when the clamp opening (130) is about 1.00 inch, the squeeze-to-set medical clamp (100) exerts a clamping force of about 1.00 lbf.

12. The squeeze-to-set medical clamp (100) of claim 1, wherein the clamp opening (130) has a range of about 0.20 inch to about 1.80 inches, and wherein the squeeze-to-set medical clamp (100) exerts a clamping force in a range of about 0.15 lbf to about 1.40 lbf within the clamp opening (130) range of about 0.20 inch to about 1.80 inches.

13. The squeeze-to-set medical clamp (100) of claim 1, wherein the squeeze-to-set medical clamp (100) has a minimum clamping force and a maximum clamping force, and wherein the minimum clamping force is at least ten percent of the maximum clamping force.

14. The squeeze-to-set medical clamp (100) of claim 1, wherein the lower jaw width (550) is at least thirty percent of the top jaw width (450).

15. A squeeze-to-set medical clamp (100) having a first unset position and a second set position, comprising:
a) a primary arm (200) having a primary arm distal end (210), a primary arm proximal end (220), a primary arm width (250), a primary arm gripping portion (260), a primary jaw connector portion (270), and a primary arm opening stop guide (280);
b) a secondary arm (300) having a secondary arm distal end (310), a secondary arm proximal end (320), a secondary arm width (350), a secondary arm gripping portion (360), a secondary jaw connector portion (370), and a secondary arm opening stop guide (380);
c) a resilient arm connector (900) joining the primary arm (200) at the primary arm proximal end (220) to the secondary arm (300) at the secondary arm proximal end (320);
d) a top jaw (400) joined to the primary arm (200) by the primary jaw connector portion (270), wherein the top jaw (400) includes a top jaw distal end (410), a top jaw proximal end (420), a top jaw dextral side (430), a top jaw sinistral side (440), a top jaw width (450), a top jaw centerline (460), a top jaw radius of curvature (470), a top jaw contact surface (480) having a top jaw contact surface center (482), and a top jaw stop ledge (490) having a ledge width (492);
e) a lower jaw (500) joined to the secondary arm (300) by the secondary jaw connector portion (370), wherein the lower jaw (500) includes a lower jaw distal end (510), a lower jaw proximal end (520), a lower jaw dextral side (530), a lower jaw sinistral side (540), a lower jaw width (550), a lower jaw centerline (560), a lower jaw radius of curvature (570), and a lower jaw contact surface (580) having a lower jaw contact surface center (582);
f) a clamp opening (130) defined by the distance between the top jaw contact surface center (482) and the lower jaw contact surface center (582); and
g) a deflector (600) positioned on at least one of the top jaw (400) and lower jaw (500), wherein the deflector (600) has a deflector height (610), a deflector width (620), and a deflector angle (630) of at least 30 degrees; and
wherein the squeeze-to-set medical clamp (100) is transitioned from the first unset position to the second set position by the steps of:
(i) applying a compressive force to the primary arm (200) and the secondary arm (300) such that at least one of the top jaw (400) and lower jaw (500) contact and translate along the deflector (600) until the top jaw (400) and lower jaw (500) are deflected and the top jaw contact surface (480) and the lower jaw contact surface (580) are in adjacent facing relationship to each other; and
(ii) removing the compressive force applied to the primary arm (200) and the secondary arm (300) such that the resilient arm connector (900) biases the top jaw (400) and the lower jaw (500) toward one another; and
wherein the top jaw stop ledge (490) engages a portion of the secondary arm (300) to prevent unintentional movement of the squeeze-to-set medical clamp (100) from the second set position to the first unset position.

16. The squeeze-to-set medical clamp (100) of claim 15, wherein the squeeze-to-set medical clamp (100) has a minimum clamping force and a maximum clamping force, and wherein the minimum clamping force is at least ten percent of the maximum clamping force.

17. The squeeze-to-set medical clamp (100) of claim 15, further including a pinch deterrent portion (800) having a pinch deterrent portion distal end (810), a pinch prevention proximal end (820), and a pinch deterrent portion contact surface (830), wherein the pinch deterrent portion distal end (810) is joined to the top jaw proximal end (420), and wherein the top jaw contact surface (480), the lower jaw contact surface (580), and the pinch deterrent portion contact surface (830) form a partially bounded circumference totaling at least 225 degrees when the squeeze-to-set medical clamp (100) is at a maximum clamp opening (130).

18. The squeeze-to-set medical clamp (100) of claim 15, wherein the deflector (600) is positioned on the top jaw (400) and the deflector width (620) extends from the top jaw sinistral side (440) to at least the lower jaw centerline (560).

19. The squeeze-to-set medical clamp (100) of claim 18, wherein the deflector width (620) is greater than or equal to a maximum lower jaw width (550).

20. A squeeze-to-set medical clamp (100) having a first unset position and a second set position, comprising:
a) a primary arm (200) having a primary arm distal end (210), a primary arm proximal end (220), a primary arm width (250), a primary arm gripping portion (260), a primary jaw connector portion (270), and a primary arm opening stop guide (280);
b) a secondary arm (300) having a secondary arm distal end (310), a secondary arm proximal end (320), a secondary arm width (350), a secondary arm gripping portion (360), a secondary jaw connector portion (370), and a secondary arm opening stop guide (380);
c) a resilient arm connector (900) joining the primary arm (200) at the primary arm proximal end (220) to the secondary arm (300) at the secondary arm proximal end (320);
d) a top jaw (400) joined to the primary arm (200) by the primary jaw connector portion (270), wherein the top jaw (400) includes a top jaw distal end (410), a top jaw proximal end (420), a top jaw dextral side (430), a top jaw sinistral side (440), a top jaw width (450), a top jaw centerline (460), a top jaw radius of curvature (470), a top jaw contact surface (480) having a top jaw contact surface center (482), and a top jaw stop ledge (490) having a ledge width (492);
e) a lower jaw (500) joined to the secondary arm (300) by the secondary jaw connector portion (370), wherein the lower jaw (500) includes a lower jaw distal end (510), a lower jaw proximal end (520), a lower jaw dextral side (530), a lower jaw sinistral side (540), a lower jaw width (550), a lower jaw centerline (560), a lower jaw radius of curvature (570), and a lower jaw contact surface (580) having a lower jaw contact surface center (582);
f) a clamp opening (130) defined by the distance between the top jaw contact surface center (482) and the lower jaw contact surface center (582); and
g) a deflector (600) positioned on at least one of the top jaw (400) and lower jaw (500), wherein the deflector (600) has a deflector height (610), a deflector width (620), and a deflector angle (630) of at least 30 degrees; and
wherein the squeeze-to-set medical clamp (100) is transitioned from the first unset position to the second set position by the steps of:
(i) applying a compressive force of about 3.00 lbf to about 6.00 lbf to the primary arm (200) and the secondary arm (300) such that at least one of the top jaw (400) and lower jaw (500) contact and translate along the deflector (600) until the top jaw (400) and lower jaw (500) are deflected and the top jaw contact surface (480) and the lower jaw contact surface (580) are in adjacent facing relationship to each other; and
(ii) removing the compressive force applied to the primary arm (200) and the secondary arm (300) such that the resilient arm connector (900) biases the top jaw (400) and the lower jaw (500) toward one another; and
wherein the squeeze-to-set medical clamp (100) in the second position exerts a clamping force in a range of about 0.15 lbf to about 0.30 lbf;
wherein when the clamp opening (130) is about 1.00 inch, the squeeze-to-set medical clamp (100) exerts a clamping force of about 1.00 lbf; and
wherein the top jaw stop ledge (490) engages a portion of the secondary arm (300) to prevent unintentional movement of the squeeze-to-set medical clamp (100) from the second set position to the first unset position.
